(19) United States
(12) Patent Application Publication
(10) Pub. No.: US 2021/0186999 A1
(43) Pub. Date: Jun. 24, 2021

(54) METHOD FOR TREATMENT OF CANCER

(71) Applicant: National University of Ireland Galway, Galway (IE)

(72) Inventors: Michael O'Dwyer, Galway (IE); Thomas Ritter, Galway (IE); Aideen Ryan, Galway (IE); Kevin Lynch, Co Louth (IE)

(21) Appl. No.: 16/760,379

(22) PCT Filed: Oct. 31, 2018
(86) PCT No.: PCT/EP2018/079902

§ 371 Date: Apr. 29, 2020

(30) Foreign Application Priority Data
Oct. 31, 2017 (GB) 1717945.8

(51) Int. Cl. A61K 31/7024; A61K 47/68; A61P 35/00; C07K 16/28

(52) U.S. Cl. CPC A61K 31/7024; A61K 47/6849; C07K 16/2851; A61K 47/6835; A61P 35/00

(57) ABSTRACT

A method of treating cancer is provided wherein the method comprises inhibiting or reducing sialylation of specifically Mesenchymal Stromal Cells (MSCs) to inhibit MSC immunosuppression and restore T cell proliferation in cancer. The method may comprise administering a sialyltransferase inhibitor (e.g. 3Fax-Peracetyl Neu5Ac) or sialidase. Also described is administration of MSCs which have been manipulated prior to administration to remove sialic acids and use of a small molecule or blocking antibody which blocks interactions between MSC-sialic acid and lectins, such as Siglec 7, and/or blocks interactions between MSC-lectins and sialic acid.

### CROSS REFERENCE TO RELATED APPLICATIONS

The present application is a U.S. National Stage Application under 35 U.S.C. § 371 of International Patent Application No. PCT/EP2018/079902, filed on Oct. 31, 2018, which claims the benefit of priority to United Kingdom Patent Application No. 1717945.8, filed on Oct. 31, 2017, the disclosure of each of which is incorporated by reference herein in its entirety.

### FIELD OF THE INVENTION

The present invention relates to a method for treating cancer. In particular, the invention relates to reversing the immunosuppressive component of the tumour microenvironment in cancer, including restoring proliferation of T cells. The immune activating approach described herein has broad applicability to many cancers.

### BACKGROUND OF THE INVENTION

Sialylation is a process by which sialic acid groups are introduced onto molecules such as oligosaccharides and carbohydrates as terminal monosaccharides. Sialic acid is a general term for N or O substituted derivatives of neuraminic acid (a 9-carbon backbone monosaccharide) which are widely expressed terminal carbohydrates on cell surface glycoproteins and glycolipids of eukaryotic cells. There are two common mammalian sialic acids: N-acetylneuraminic acid (Neu5Ac) and N-glycolylneuraminic acid (Neu5Gc), which are synthesised by four consecutive reactions from UDP-N-acetylglucosamine. Due to their abundant presence on the surface of eukaryotic cells, sialic acids play key roles in several pathophysiological processes including metastasis, tumour progression, inflammation and viral infection. The amount of sialic acid is governed by levels of sialidases and sialyltransferases.

Sialyltransferases are enzymes that transfer sialic acid to nascent oligosaccharide. Each sialyltransferase is specific for a particular sugar substrate. Sialyltransferases add sialic acid to the terminal portions of the sialylated glycolipids (gangliosides) or to the N- or O-linked sugar chains of glycoproteins. There are about twenty different sialyltransferases which can be distinguished on the basis of the acceptor structure on which they act and on the type of sugar linkage they form. Each of these sialyltransferase genes is differentially expressed in a tissue-, cell type-, and stage-specific manner to regulate the sialylation pattern of cells. They differ in their substrate specificity, tissue distribution and various biochemical parameters. For example, one group of sialyltransferases adds sialic acid with an alpha-2,3 linkage to galactose, while other sialyltransferases add sialic acid with an alpha-2,6 linkage to galactose or N-acetylgalactosamine.

Sialidase is one of the most important enzymes of the sialic acid catabolism causing removal of sialic acid residues from the cell surface or serum sialoglycoconjugates.

Usually, in higher animals, the glycoconjugates that are prone to be degraded are captured by endocytosis. After the fusion of the late endosome with the lysosome, lysosomal sialidases remove sialic acid residues. The activity of these sialidases is based on the removal of O-acetyl groups. Free sialic acid molecules are transported to the cytosol through the membrane of the lysosome. There, they can be recycled and activated again to form another nascent glycoconjugate molecule in the Golgi apparatus. Sialidases, e.g. NEU-1, can also act at the level of the cell membrane (Lukong K E, Seyrantepe V, Landry K, Trudel S, Ahmad A, Gahl W A, Lefrancois S, Morales C R, Pshezhetsky A V. Intracellular distribution of lysosomal sialidase is controlled by the internalization signal in its cytoplasmic tail. J Biol Chem. 2001 Dec. 7; 276(49):46172-81. Epub 2001 Sep. 24. PubMed PMID: 11571282).

Tumorigenesis and metastasis are frequently associated with altered structure and expression of oligosaccharides on cell surface glycoproteins and glycolipids of tumour cells. The expression of sialylated glycoconjugates on tumour cells has been shown to change during development, differentiation, disease and oncogenic transformation. Abnormal sialylation in cancer cells is a distinctive feature associated with malignant properties including invasiveness and metastatic potential. Methods of targeting sialylation of tumour cells are being investigated as a means for treating cancer. For example, European Patent Publication No. 2906952A discloses the use of a sialyltransferase inhibitor to target myeloma cells to treat Multiple Myeloma.

Mesenchymal Stromal Cells (MSCs) are multipotent stromal cells found in bone marrow that can differentiate into a variety of cell types, including osteoblasts, chondrocytes, myocytes and adipocytes. MSCs are potent suppressors of the immune system and it is now recognized that immune suppression within the tumour microenvironment is a major reason for failure of anti-cancer therapy. Immune checkpoint inhibitory drugs are being investigated as a means for overcoming immune suppression within the tumour microenvironment and several monoclonal antibodies are in development as checkpoint inhibitors. However, these need to be produced by genetic engineering and are therefore expensive to produce. Moreover, they have a single mode of action. A new means for overcoming the immune suppressive phenotype of the tumour microenvironment would therefore be advantageous in obtaining a beneficial anti-cancer response that is applicable to many cancers.

### SUMMARY OF THE INVENTION

The present inventors have identified a link between sialylation of Mesenchymal Stromal Cells (MSCs) and their immunosuppressive properties. In particular, inhibition of sialylation of MSCs is shown by the inventors to restore proliferation of T cells, thus overcoming an important immunosuppressive component of the tumour microenvironment in cancer. This provides a highly cost effective approach to reversing immune suppression in patients with cancer and the immune activating approach described herein has broad applicability to many cancers as immunosuppression is a common mechanism in a cancer setting.

Accordingly, according to a first aspect of the present invention there is provided an inhibitor of sialylation for use in treatment of cancer wherein the inhibitor is targeted to inhibit or reduce sialylation of specifically Mesenchymal Stromal Cells (MSCs).

Typically the inhibition or reduction of sialylation of MSCs upregulates T cells and/or natural killer (NK) cells.

Typically cell surface sialylation of MSCs may be reduced or inhibited.

Typically the inhibitor is a sialyltransferase inhibitor. The use of a sialyltransferase inhibitor avoids problems associated with drug resistance and may assist in inhibiting metastases.

Sialyltransferases may be specific to different tissue and cell types and may differ in their substrate specificity. In certain embodiments, targeting the sialyltransferase inhibitor to inhibit sialylation of specifically MSCs comprises using a sialyltransferase inhibitor which specifically inhibits sialyltransferases associated with or specific to MSCs. The use of a selective sialyltransferase inhibitor that would only target the sialyltransferases of relevance in the MSCs would be advantageous as it would avoid any risk of off target effects that could occur with a pan sialyltransferase inhibitor. The sialyltransferase inhibitor may be administered using a systemic route of administration.

In certain embodiments, the sialyltransferase inhibitor inhibits sialyltransferases that catalyse the addition of sialic acid onto sugars with alpha 2, 3 sugar linkages. In certain embodiments, the sialyltransferase inhibitor inhibits sialyltransferases that catalyse the addition of sialic acid onto sugars with alpha 2, 6 sugar linkages. In certain embodiments, the sialyltransferase inhibitor inhibits sialyltransferases that catalyse the addition of sialic acid onto sugars with alpha 2, 8 sugar linkages.

In certain embodiments, targeting the sialyltransferase inhibitor to inhibit sialylation of specifically MSCs comprises using a sialyltransferase inhibitor that is targeted for delivery to specifically bone marrow microenvironment. In certain embodiments, the sialyltransferase inhibitor is targeted for delivery to specifically MSCs. Selective delivery to the bone marrow or MSCs reduces the risk of off-target toxicity, in particular nephrotoxicity. The sialyltransferase inhibitor may be a pan sialyltransferase inhibitor (e.g. 3Fax-Peracetyl Neu5Ac) or a selective sialyltransferase inhibitor that would only target the sialyltransferases of relevance in the MSCs as described above. The sialyltransferase inhibitor may be administered using a systemic route of administration. Targeted delivery to the bone marrow microenvironment where MSCs reside or to the MSCs themselves may be achieved by conjugating the sialyltransferse inhibitor to monoclonal antibodies that target antigens specific to the bone marrow microenvironment or MSCs. Examples of MSC specific antigen include CD105, CD90 and CD73. MSCs lack expression of CD45, CD34, CD14 or CD11b, CD79alpha or CD19 and HLA-DR. The sialyltransferase inhibitor may be formulated as nanoparticles. The nanoparticles may be coated with the monoclonal antibody. Inhibition of sialylation requires uptake and internalisation of the sialyltransferase inhibitor by MSC's to block action of sialyltransferases in the Golgi. The sialyltransferase inhibitor gradually inhibits cell surface sialylation by preventing incorporation of new sialic acid residues onto elongating glycans on lipids or proteins. In certain embodiments, the sialyltransferase inhibitor is 3Fax-Peracetyl Neu5Ac.

In certain embodiments, the sialyltransferase inhibitor is selected from the group consisting of (i) sialic acid analogs, (ii) CMP-sialic acid analogs, (iii) cytidine analogs, (iv) oligosaccharide derivatives, (v) aromatic compounds, (vi) flavonoids and (vii) lithocholic acid analogs. In certain embodiments, the sialyltransferase inhibitor is a sialic acid analog, for example, 3Fax-Peracetyl Neu5Ac. In certain embodiments, the sialyltransferase inhibitor is a CMP-sialic acid analog, for example, cytidin-5′-yl sialylethylphosphonate. In certain embodiments, the sialyltransferase inhibitor is a cytidine analog, for example, 5-methyl CMP or 2′-O-methyl CMP. In certain embodiments, the sialyltransferase inhibitor is an aromatic compound, for example, a negatively charged sulfonic acid analog which is a natural aromatic compound with sialyltransferase inhibition attributes. Further examples of sialyltransferase inhibitors are described in Wang L, Liu Y, Wu L, Sun X-L. Sialyltransferase inhibition and recent advances. Biochimica et Biophysica Acta 1864 (2016) 143-153.

In certain embodiments, the inhibitor is sialidase. Accordingly, according to a second aspect of the present invention there is provided sialidase for use in treatment of cancer wherein the sialidase is targeted specifically to Mesenchymal Stromal Cells (MSCs).

In certain embodiments, targeting the sialidase specifically to MSCs comprises using a sialidase that is targeted for delivery to specifically bone marrow microenvironment. In certain embodiments, the sialidase is targeted for delivery to specifically MSCs. Sialidase cleaves sialic acid residues at specific sugar linkages on cell surface glycoproteins or glycolipids. Targeted delivery to the bone marrow microenvironment where MSCs reside or to the MSCs themselves may be achieved by conjugating the sialidase to monoclonal antibodies that target antigens or cell surface markers specific to the bone marrow microenvironment or MSCs, for example, as described above. The sialidase may be formulated as nanoparticles. The nanoparticles may be coated with the monoclonal antibody. In certain embodiments, the sialidase is NEU-1.

According to a third aspect of the present invention there is provided Mesenchymal Stromal Cells (MSCs) for use in treatment of cancer in a subject wherein the MSCs have been manipulated to remove sialic acids prior to administration to the subject.

Where MSCs are infused in cancer patients for therapeutic purposes, the MSCs may be manipulated to remove sialic acids prior to infusion. This could be achieved using, for example, small interfering RNAs or Crispr/Cas9 technologies. Preferably, the manipulation of the MSCs will not affect the homing properties of the MSCs—in this respect, preferably alpha 2,3 linked sialic acids involved in Sialyl Lewis X synthesis will not be removed, for example, alpha 2,3 linked sialic acids generated by ST3GAL4 and ST3GAL6. In that case, manipulating the MSCs may comprise removing other alpha 2,3 linked sialic acids, such as ST3GAL1, ST3GAL5, that may be important in MSC sialylation, but not important in generation of Sialyl Lewis, and/or removing alpha 2,6 linked sialic acids.

According to a fourth aspect of the present invention there is provided a small molecule or blocking antibody that blocks interactions between Mesenchymal Stromal Cell (MSC)-sialic acids and lectins and/or between MSC-lectins and sialic acid for use in the treatment of cancer.

In certain embodiments, the small molecule or blocking antibody blocks interactions between MSC-sialic acids and lectins on immune cells, such as NK cells, for example, siglecs such as Siglec 7 or Siglec 9. The small molecule or blocking antibody may additionally or alternatively block interactions between sialic acids (for example, on immune cells, such as NK cells) and lectins on MSCs, for example, siglecs such as Siglec 7 or Siglec 9. The small molecule may comprise a sialic acid mimetic. The blocking antibody may be a monoclonal antibody, e.g. a monoclonal antibody that blocks Siglec-Siglec ligand interactions. In certain embodiments, the small molecule or blocking antibody may block interactions between sialic acids and Siglec 7. In certain embodiments, the small molecule or blocking antibody does not block interactions between sialic acids and Siglec 9.

According to a further aspect of the present invention there is provided a method of treating cancer in a subject in need thereof, the method comprising inhibiting or reducing sialylation of specifically Mesenchymal Stromal Cells (MSCs) of the subject.

The method may comprise administering an inhibitor of sialylation, in particular, a sialyltransferase inhibitor or sialidase, to the subject. The inhibitor may be a sialyltransferase inhibitor or sialidase as described above. The method may further comprise administering MSCs to the subject wherein the MSCs have been manipulated to remove sialic acids prior to administration to the subject, for example, as described above.

Also provided is a method of treating cancer in a subject in need thereof, the method comprising administering a small molecule or blocking antibody that blocks interaction between Mesenchymal Stromal Cell (MSC)-sialic acid and lectins and/or between MSC-lectins and sialic acids. The small molecule or blocking antibody may be a small molecule or blocking antibody as described above.

Further provided is use of a sialyltransferase inhibitor, sialidase, manipulated MSCs, a small molecule or a blocking antibody as described above in the preparation of a medicament for the treatment of cancer.

### BRIEF DESCRIPTION OF THE FIGURES

The invention will be more clearly understood from the following description of some embodiments thereof, given by way of example only, with reference to the following figures in which:

FIGS. 1(a)-1(c) show sialic acid is upregulated in (a) a pro-inflammatory environment, (b) Multiple Myeloma and (c) colon cancer. CM=Multiple myeloma conditioned media. MWS=multiple myeloma conditioned media without fetal bovine serum. TCM=Colon cancer conditioned media. Statistics: Unpaired T test and one-way ANOVA where applicable P<0.05*<0.005**<0.0005***.

FIGS. 2(a)-2(f) show pre-treated MSCs have heightened T-cell immunosuppression. FIG. 2(a) shows CD4+ Lymphocytes, FIG. 2(b) shows total CD4+ proliferation, FIG. 2(c) shows CD8+ Lymphocytes, FIG. 2(d) shows total CD8+ proliferation, FIG. 2(e) shows levels of NaNO₂, and FIG. 2(f) shows PGE2 levels.

FIGS. 3(a)-3(c) show inhibition of glycosylation impairs MSCs immunomodulation.

FIG. 3(a) shows CD4⁺ Lymphocytes, FIG. 3(b) shows total CD4⁺ proliferation,

FIG. 3(c) shows CD8⁺ Lymphocytes and FIG. 3(d) shows total CD8⁺ proliferation.

FIGS. 4(a)-4(c) show inhibition of glycosylation impairs MSCs immunomodulation, but that this is NO (FIG. 4(a)), IL-10 FIG. 4(b)) and PGE2 FIG. 4(c)) independent.

FIGS. 5(a)-5(d) show hMSCs express both Siglec 7 (FIG. 5(a)) and Siglec 9 (FIG. 5(b)) receptors. In a pro-inflammatory environment, Siglec 7 is increased (TNF-α+IL-1β) compared to unstimulated MSCs (FIG. 5(a)). The opposite is seen for Siglec 9 (FIG. 5(b)).

FIGS. 6(a)-6(b) show multiple myeloma Mesenchymal Stromal Cells (MM MSCs) have increased sialylation profiles compared to healthy MSC controls. The sialic acid profiles of both MM MSCs and healthy MSCs were analysed by flow cytometry.

FIGS. 7(a)-7(c) show MM MSCs have an increased ability to supress activated T lymphocytes. Both MM MSCs and healthy MSCs were placed into T lymphocyte co-cultures for 96 hours. FIG. 7(a) shows % CD4 proliferation, FIG. 7(b) shows CD4 Counts, FIG. 7(c) shows % CD8 proliferation, and FIG. 7(d) shows CD8 Counts.

FIGS. 8(a)-8(b) show MM MSCs have an increased ability to supress activated Macrophages. Both MM MSCs and healthy MSCs were placed into macrophage co-cultures for 72 hours in the presence of IFN-γ and LPS. MM MSCs had an increased ability to supress the activated phenotype of IFN-γ stimulated macrophages.

### DETAILED DESCRIPTION OF THE INVENTION

The present inventors have identified that inhibition of sialylation of Mesenchymal Stromal Cells (MSCs) reduces the immunosuppressive properties of MSCs. The immunosuppressive component of MSCs is a common feature in cancer and the present invention therefore has broad applicability to cancer in general, effectively acting as a new checkpoint inhibitor.

Previous attempts focusing on sialylation in cancer were aimed at inhibiting sialylation of specifically tumour cells using, for example, labelled antibodies against tumour antigen. These attempts focused specifically on sialylation of tumour cells as off target toxicity was considered a problem. The present inventors have identified a link between sialylation in specifically MSCs and immunosuppression in cancer, resulting in treatments which target specifically MSCs. Selective delivery to the bone marrow or MSCs reduces the risk of off-target toxicity, in particular nephrotoxicity.

Inhibition of sialylation of MSCs is shown by the inventors to restore proliferation of T cells, thus overcoming an important immunosuppressive component of the tumour microenvironment in cancer. Without wishing to be bound by theory, a reduction in sialylation of MSCs may also polarize macrophages, which could have beneficial effects independent of T cell proliferation, such as on Natural Killer (NK) cells. For example, if macrophages express less PDL-1, then there may be less of an inhibitory effect on NK cells, which can express PD-1.

The inventors have also shown an upregulation of Siglec-7 on MSCs in an inflammation setting. Siglec-7 is known to play an important role in negative regulation of T cells (Ikehara Y, Ikehara S K, Paulson J C. Negative regulation of T cell receptor signaling by Siglec-7 (p70/AIRM) and Siglec-9. J Biol Chem. 2004 Oct. 8; 279(41):43117-25. Epub 2004 Aug. 3. PubMed PMID: 15292262). This could be an important link between MSCs and T cells in inflammation.

Also, without wishing to be bound by theory, siglec ligands on MSCs could directly interact with siglecs (e.g. Siglec 7) on NK cells, causing inhibition of NK cells. Siglec-7 is also known to play an important role in inhibiting NK cells.

Methods for inhibiting sialylation are described in International Patent Publication No. WO 2008/087256 A1. This document describes specific sialylated structures present on human stem cells and cell populations derived thereof.

A subject in need thereof may be a subject who is suffering from cancer or a cancer patient.

Treatment (e.g. sialyltransferase inhibitor, sialidase, modified MSCs, small molecule or blocking antibody) may be combined with one or more standard cancer treatments. The treatment may be administered alone or may be administered as a pharmaceutical composition which will generally comprise a suitable pharmaceutically acceptable excipient, diluent or carrier. The pharmaceutically acceptable excipient, diluent or carrier may be selected depending on the intended route of administration. Examples of suitable pharmaceutical carriers include water, glycerol and ethanol.

The treatment may be administered to a patient in need of treatment via any suitable route. The treatment may be administered parenterally by injection or infusion. Examples of preferred routes for parenteral administration include, but are not limited to, intravenous, intracardial, intraarterial, intraperitoneal, intramuscular, intracavity, subcutaneous, transmucosal, inhalation and transdermal. Routes of administration may further include topical and enteral, for example, mucosal (including pulmonary), oral, nasal and rectal. The treatment may also be administered via nanoparticles, microspheres, liposomes, other microparticulate delivery systems or sustained release formulations placed in certain tissues including blood.

The treatment is typically administered to a subject in a “therapeutically effective amount”, this being an amount sufficient to show benefit to the subject to whom the treatment is administered. The actual dose administered, and rate and time-course of administration, will depend on, and can be determined with due reference to, the nature and severity of the condition which is being treated, as well as factors such as the age, sex and weight of the subject being treated, as well as the route of administration. Further due consideration should be given to the properties of the treatment, for example, its in-vivo plasma life and concentration in the formulation, as well as the route, site and rate of delivery. Prescription of treatment, e.g. decisions on dosage, etc., is ultimately within the responsibility and at the discretion of general practitioners and other medical doctors, and typically takes account of the disorder to be treated, the condition of the individual patient, the site of delivery, the method of administration and other factors known to practitioners.

Dosage regimens can include a single administration, or multiple administrative doses. The treatment can further be administered simultaneously, sequentially or separately with other therapeutics and medicaments which are used for the treatment of the cancer for which the treatment is being administered.

### Definitions

Unless otherwise defined, all technical and scientific terms used herein have the meaning commonly understood by a person who is skilled in the art in the field of the present invention.

As used herein, the term “cancer” is understood to refer to a group of diseases involving abnormal cell growth with the potential to invade or spread to other parts of the body. The cancer may, for example, be selected from the group consisting of lung cancer, prostate cancer, colorectal cancer, stomach cancer, bowel cancer, breast cancer, oral cancer, pancreatic cancer and cervical cancer.

Typically the terms “subject” and “patient” are used interchangeably herein. The subject is typically a mammal, more typically a human.

The terms “inhibit” and “inhibiting” are used herein to refer to both partial inhibition (i.e. a reduction) and complete inhibition.

The term “inhibitor of sialylation” is used herein to refer to any compound or drug that inhibits or reduces sialylation by, for example, inhibiting or reducing addition of sialic acids (e.g. a sialyltransferase inhibitor) and/or promoting removal of sialic acids (e.g. sialidase).

References to “specifically Mesenchymal Stromal Cells (MSCs)”, “targeting specifically Mesenchymal Stromal Cells (MSCs)” or “targeting inhibition of sialylation of specifically MSCs” or similar are used herein to refer to the ability of the treatment (e.g. sialidase or sialyltransferase inhibitors) to inhibit or reduce sialylation of MSCs at a higher rate than sialylation of other cell types, including tumour cells, i.e. the treatment is aimed at reducing or inhibiting sialylation of primarily MSCs rather than sialylation of other cell types. In certain embodiments, there is also an effect on sialylation of other cell types, but this is lower than the effect on sialylation of MSCs, preferably significantly so. In alternative embodiments, the effect on sialylation is restricted to sialylation of MSCs. Similarly, references to “inhibiting specifically sialyltransferases associated with or specific to MSCs” or similar are used herein to refer to the ability of the treatment (e.g. sialyltransferase inhibitor) to inhibit sialyltransferases of MSCs at a higher rate than sialyltransferases of other cell types, including tumour cells, i.e. the treatment is aimed at reducing or inhibiting sialylation of primarily MSCs rather than sialylation of other cell types. In certain embodiments, there is also an effect on sialyltransferases of other cell types, but this is lower than the effect on sialyltransferases of MSCs, preferably significantly so. In alternative embodiments, the effect on sialyltransferases is restricted to sialyltransferases of MSCs.

The term “treatment” as used herein and associated terms such as “treat” and “treating” means the reduction of the progression, severity and/or duration of cancer or at least one symptom thereof. The term ‘treatment’ therefore refers to any regimen that can benefit a subject. Treatment may include curative, alleviative or prophylactic effects.

The term “bone marrow microenvironment” as used herein includes all the cellular and structural components of the bone marrow, including, but not limited to, fat cells, hematopoietic stem cells, progenitor cells and precursor cells.

The term “tumour microenvironment” as used herein refers to the cellular environment in which the tumour/cancerous cells exist.

The words “comprises/comprising” and the words “having/including” when used herein with reference to the present invention are used to specify the presence of stated features, integers, steps or components, but do not preclude the presence or addition of one or more other features, integers, steps, components or groups thereof.

As used herein, terms such as “a”, “an” and “the” include singular and plural referents unless the context clearly demands otherwise.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

### Example—Inhibition of sialylation impairs MSCs immunosuppression

Materials and Methods Four female BALB/c at approximately 5 to 7 weeks were euthanized and the femur and tibia was dissected and placed in a 50 ml tube with media or PBS. Using a forceps and scissors the bones were cleaned to remove muscles and snipped on both ends to plunge out the marrow into a Petri dish containing MSC medium. A 30G needle fitted on a 1 ml syringe was used to plunge out the marrow and repeated several times till the bones appeared white. The marrow was passed through a 40 μm sieve attached to a 50 ml tube and the sieve was washed twice with medium. Medium was changed, and a cell count was performed. 10 μl cells was mixed with 40 μl PBS and 10 μl 4% acetic acid and cell count was performed. Based on the count approximately 35-50 million cells were seeded per T175 cm flask or 1 animal per flask. After isolation and culturing the MSCs were extensively characterized in vitro before and after treatment with TNF-α+IL-1β.

Human MSCs (hMSC) were isolated from the bone marrow of three human donors. Cells were seeded in T175 flasks at 5×10⁴ mononuclear cells/cm². Cells were extensively characterised in vitro before use in experiments.

Sialic acid content was assayed using lectin coupled flow cytometry. Biotin labelled SNA-I and MAL-II were used to detect a 2-6 sialic acid and a 2-3 sialic acid respectively. Sialyltransferase inhibition was carried out using 100 μM of 3Fax-Peracetyl Neu5Ac. Cells were cultured in the presence of the inhibitor for two successive passages to ensure inhibition.

To determine if TNF-α+IL-1βMSC displayed enhanced immunoregulatory ability, they were co-cultured in mixed lymphocyte reactions (MLRs). MSC and TNF-α+IL-1βMSC were co-cultured at different MSC: T-cell ratios for 96 hrs. T-cell proliferation, activation, death and differentiation were determined by flow cytometry.

Both ELISA and the Griess assay were used to determine quantity of immunomodulatory molecules in the supernatant of mixed lymphocyte reaction experiments.

Results and Discussion

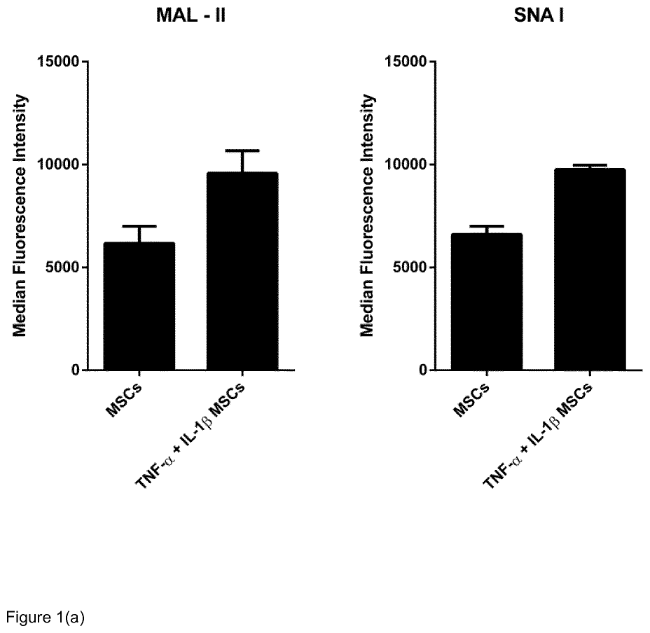

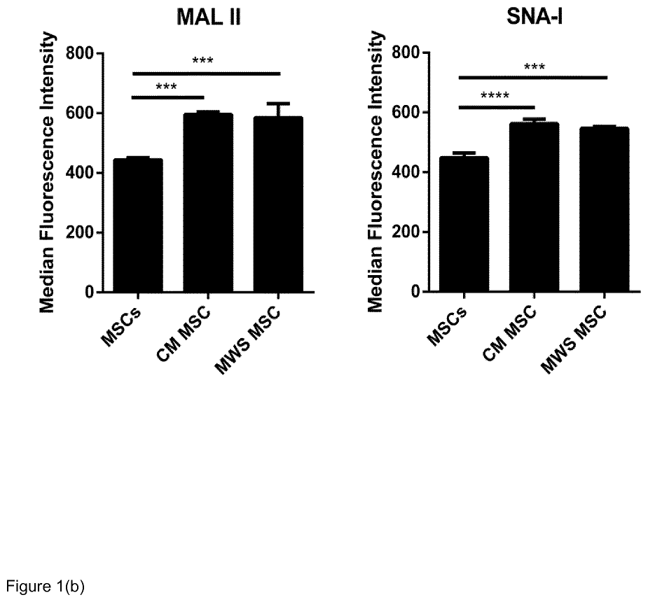

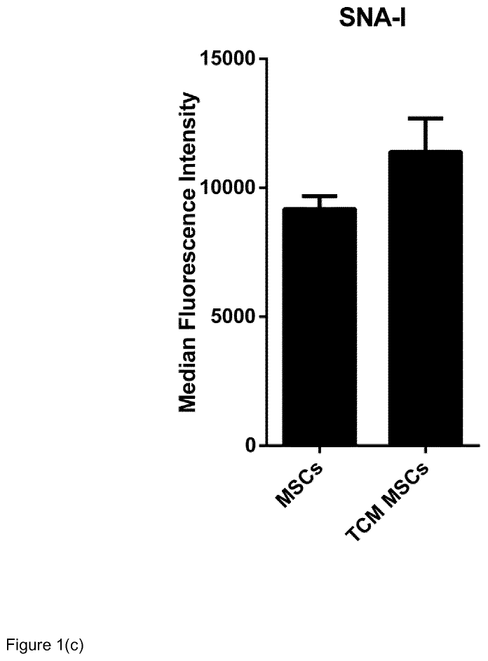

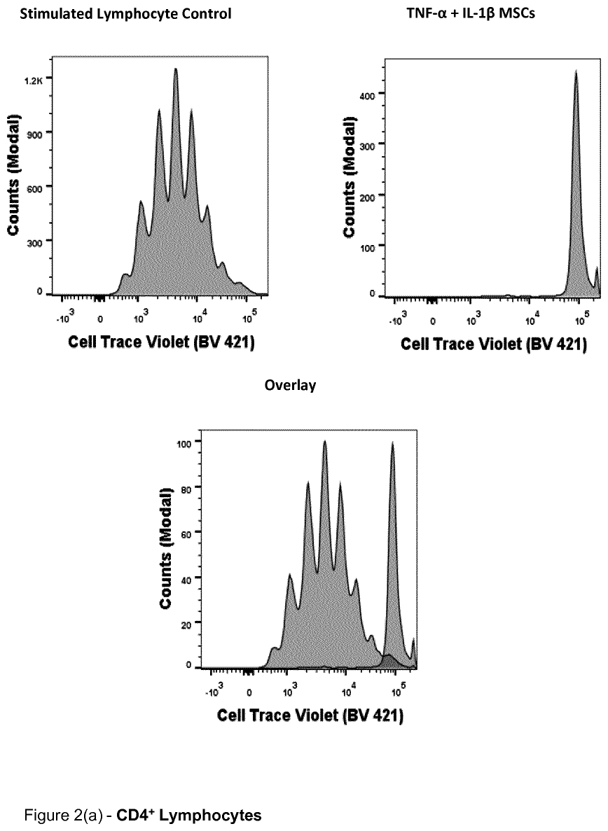

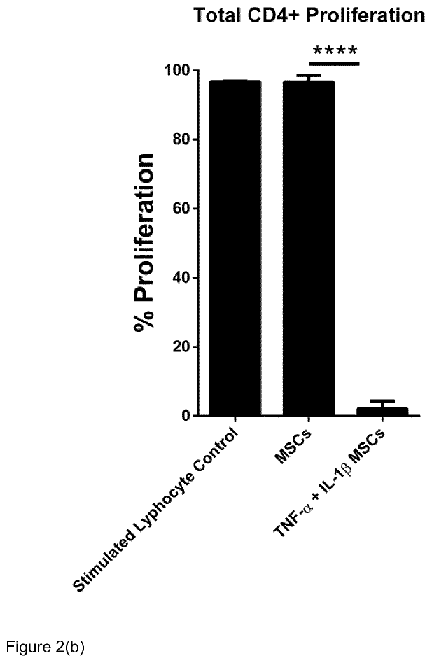

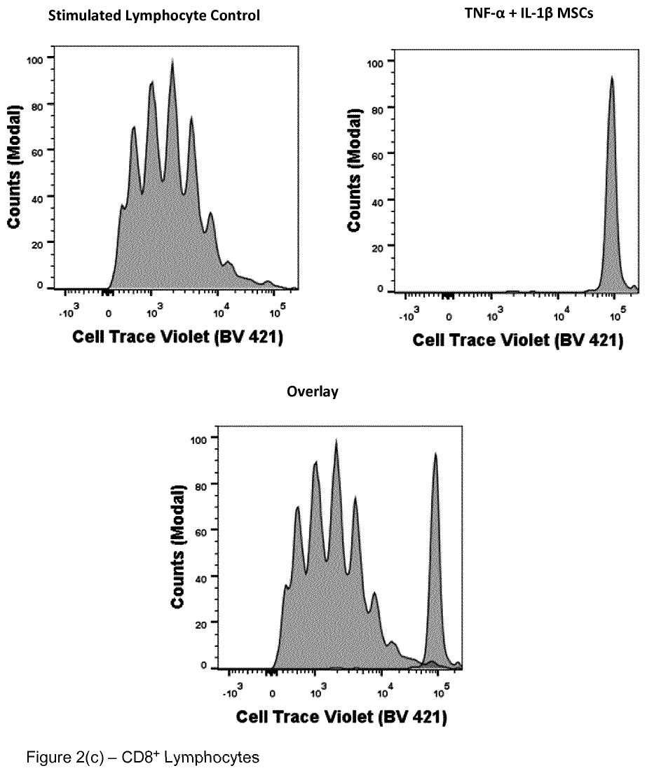

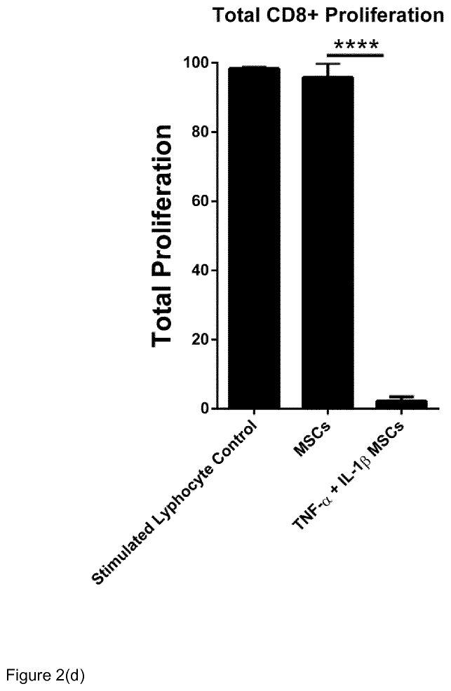

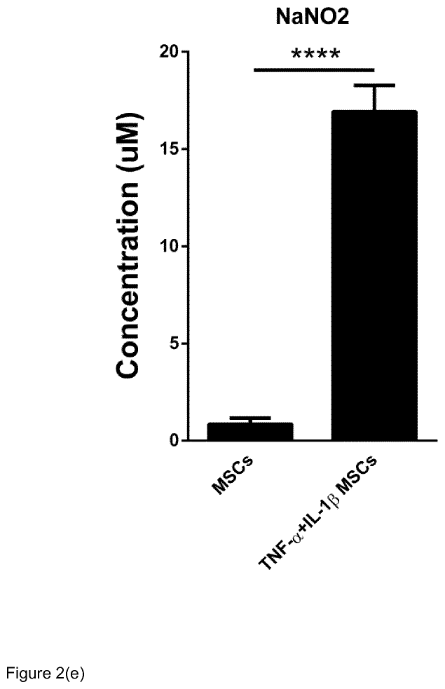

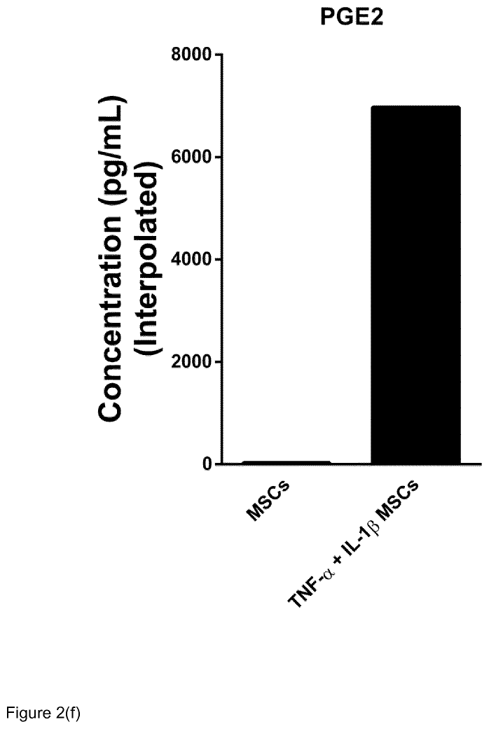

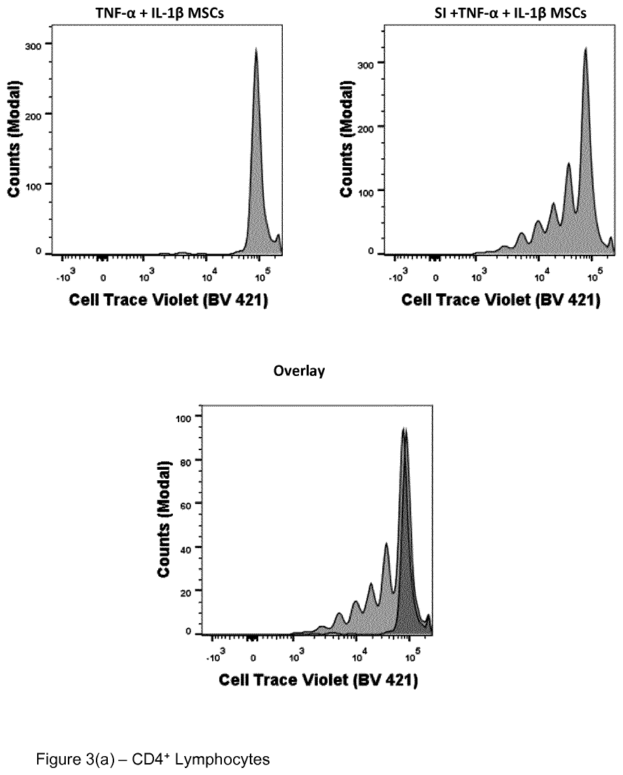

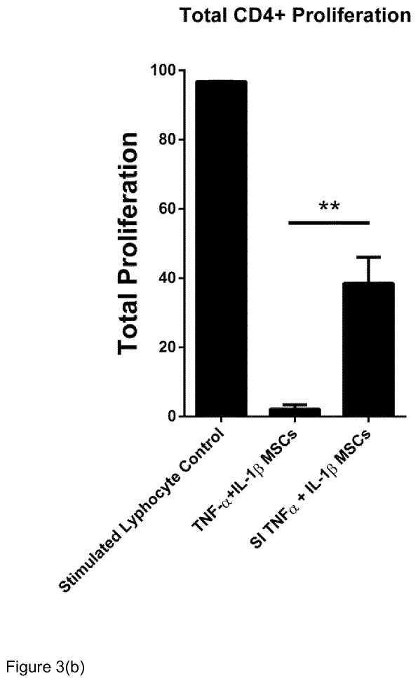

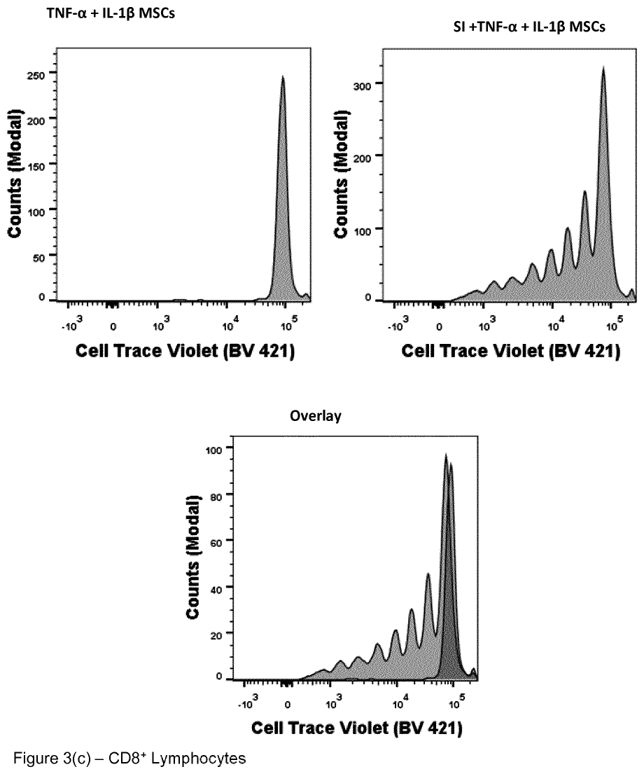

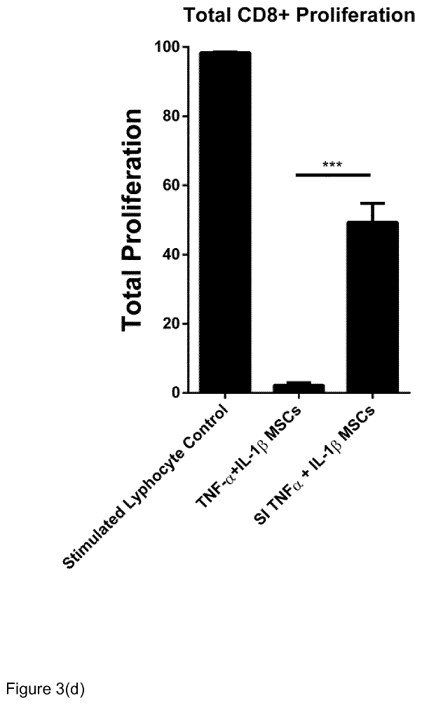

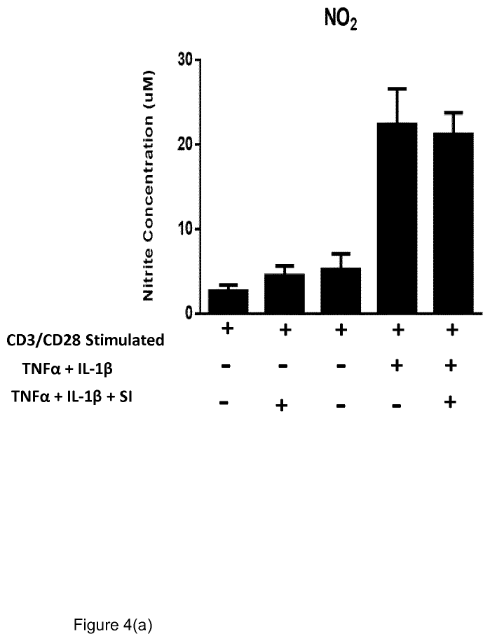

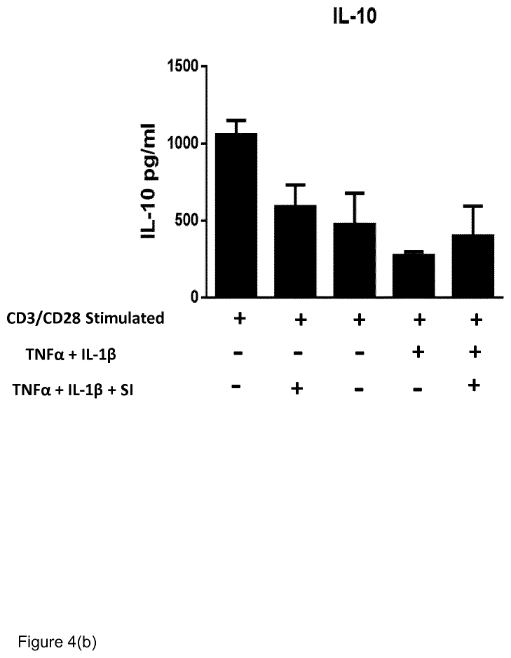

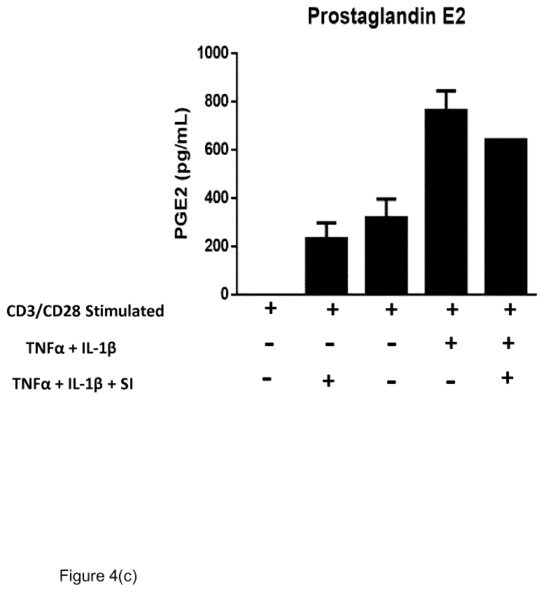

MSCs have increased T-cell immunosuppressive capacity when they are exposed to a pro-inflammatory microenvironment (FIGS. 1(a)-1(c)). This increased immunosuppressive potential is due to increases in secreted molecules such as nitric oxide (NO), prostaglandin E2 (PGE2), transforming growth factor beta 1 (TGF-β), interleukin 10 (IL-10) and cell surface programed death ligand 1 (PD-L1). In this study, the inventors investigated what role sialic acid plays in the immunosuppressive attributes of MSCs. To study what role sialic acid plays in MSC immunomodulation, the inventors used the sialyltransferase inhibitor 3Fax-Peracetyl Neu5Ac. This is a cell-permeable sialylic acid analog that is converted to a CMP-Neu5Ac and inhibits sialyltransferase. To stimulate the MSCs to be more immunosuppressive, the inventors mimicked the pro-inflammatory microenvironment by pre-treating the MSCs with the pro-inflammatory cytokines IL-1 and TNF-α, conditioning them with supernatants from multiple myeloma cell lines or conditioning them with supernatants from colon cancer cell lines. The inventors observed that sialic acid is increased under these conditions (FIGS. 1(a)-1(c)). The inventors also observed increased lymphocyte suppression from MSCs conditioned in these environments (FIGS. 2(a)-2(f)). The inventors tested the immunomodulatory capacity of MSCs treated with and without the inhibitor (FIGS. 3(a)-3(c)). TNF-α+IL-1β treated MSCs after being exposed to the sialyltransferase inhibitor were not as effective at suppressing activated T-cells (FIGS. 3(a)-3(c)). The inventors then looked to see if the restoration in proliferation was due to inhibition of one of the key proteins involved in MSC modulation (FIGS. 4(a)-4(c)). While the sialyltransferase inhibitor restored proliferation of T-cells, it seems to be independent of nitric oxide (NO), prostaglandin E2 (PGE2), and IL-10.

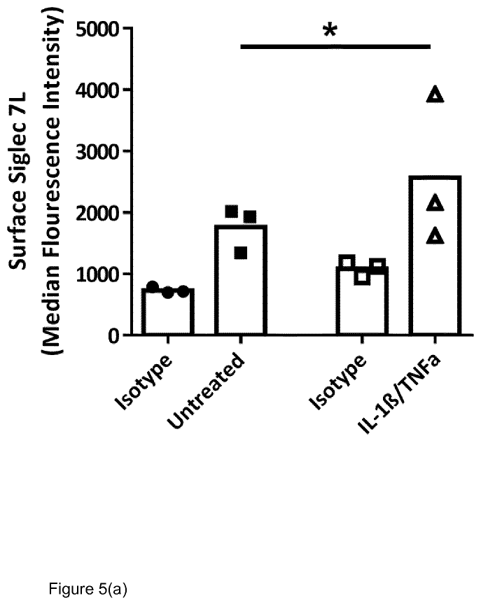

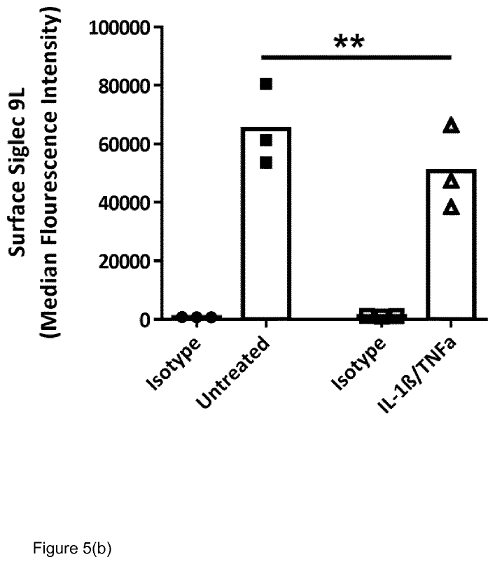

The Siglecs are a family of sialic-acid-binding immunoglobulin-like lectins that are thought to promote cell-cell interactions and regulate the functions of cells in the innate and adaptive immune systems through glycan recognition. The inventors have shown for the first time that human MSCs (hMSCs) express both Siglec 7 and Siglec 9. Three human donors showed an increase in Siglec 7 after pro-inflammatory stimulus while Siglec 9 decreases (FIGS. 5(a)-5(d)). This reinforces the importance of sialic acid content in immunomodulation of immune cells by MSCs.

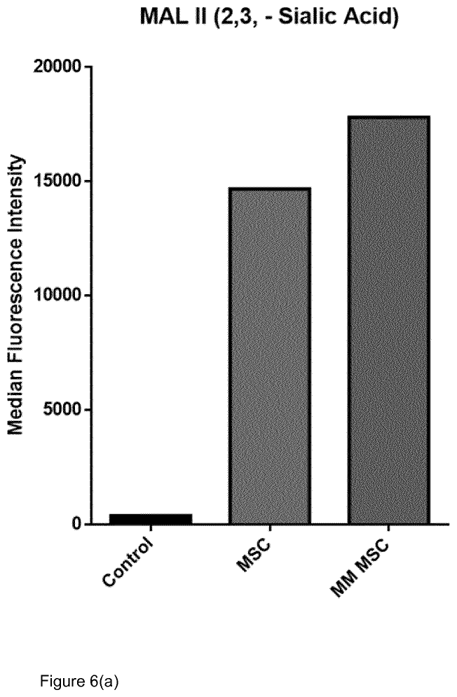

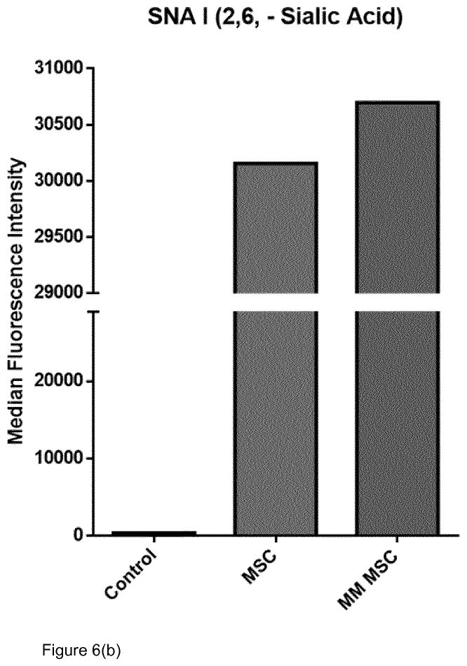

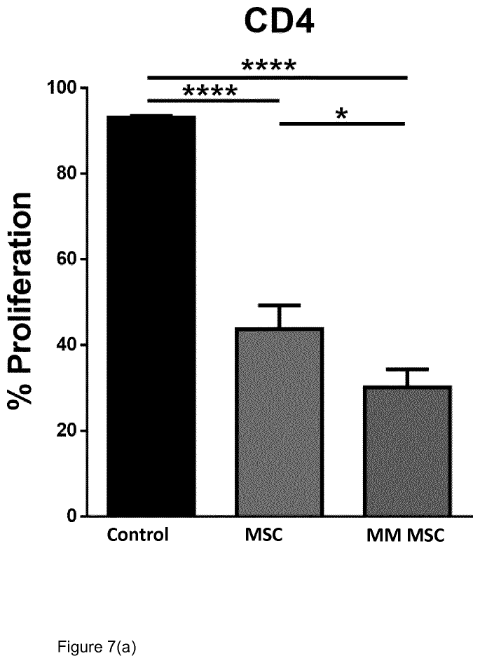

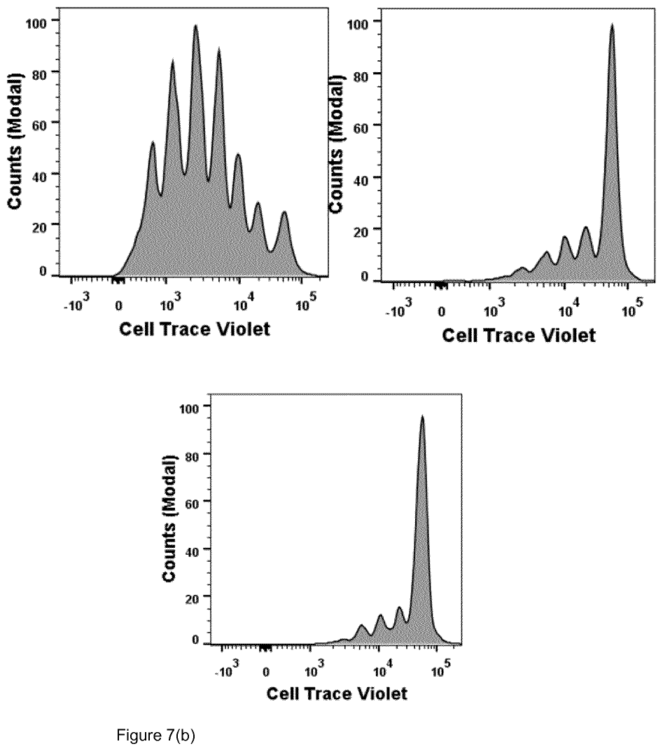

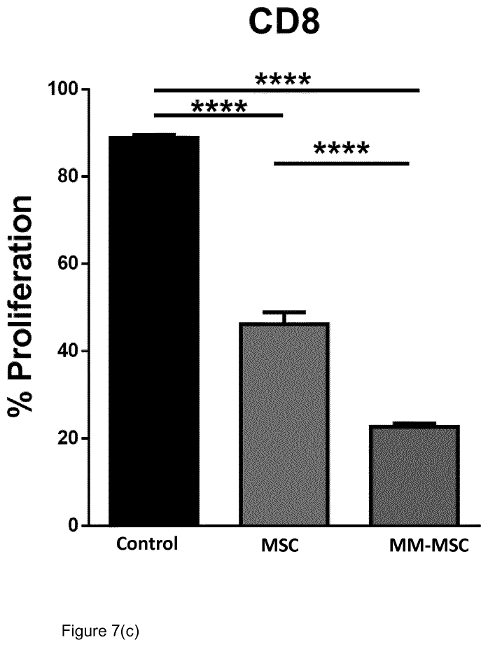

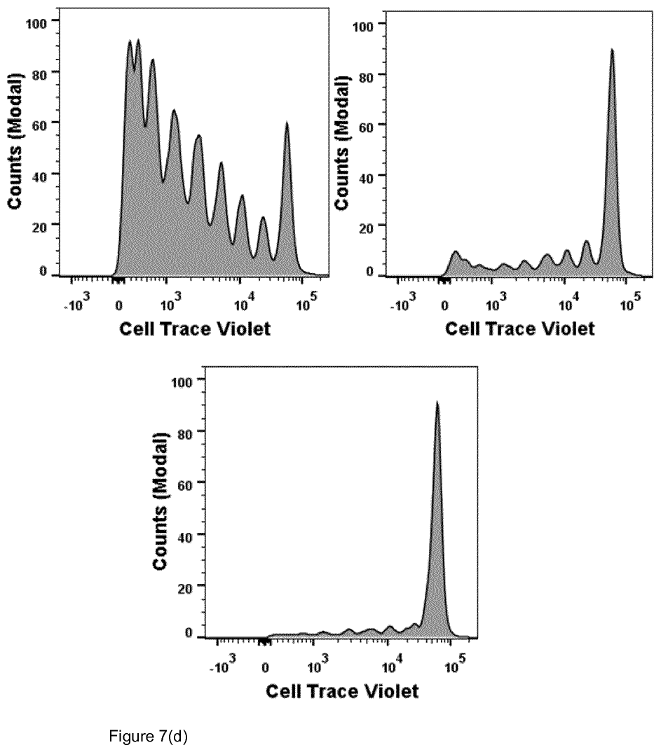

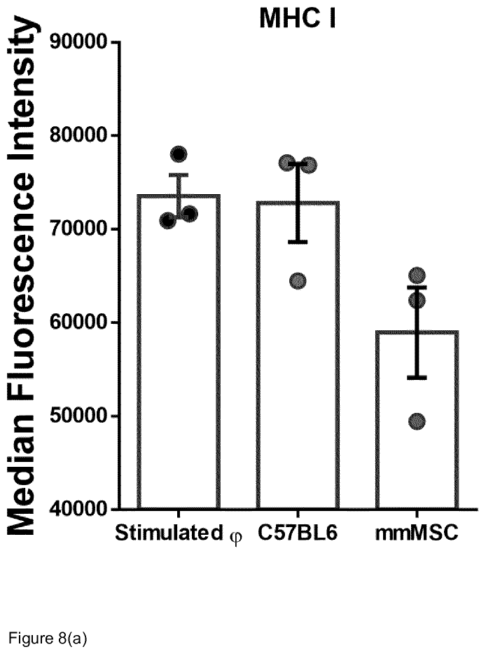

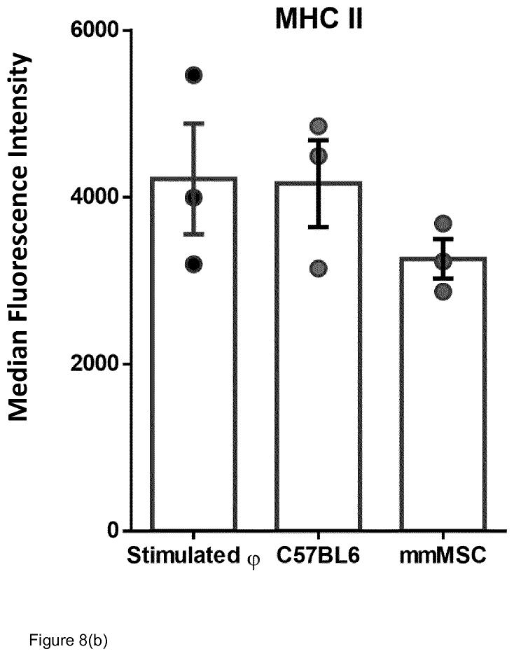

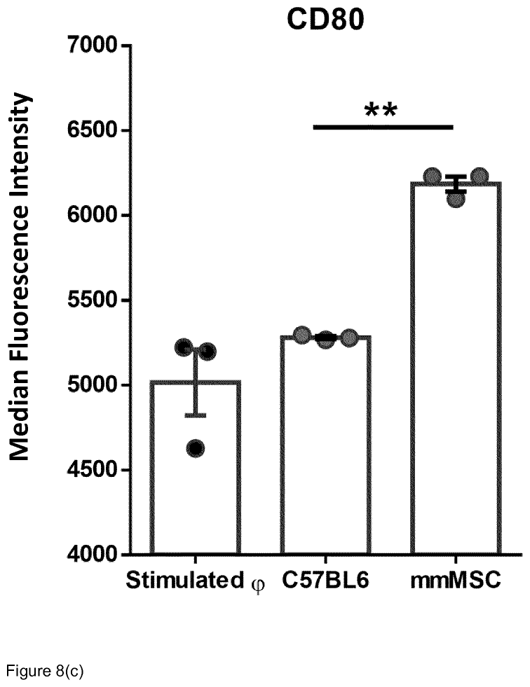

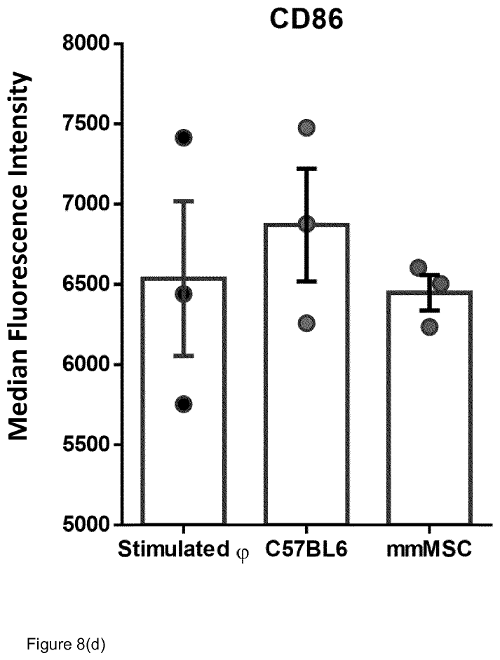

FIGS. 6(a)-6(b) show that MSCs isolated from myeloma bearing mice had increased sialic acid profiles compared to healthy controls, further supporting that sialic acid is a potential target for cancer treatment. FIGS. 7(a)-7(c) show that MM MSCs have an increased ability to supress activated T lymphocytes compared to healthy controls and FIGS. 8(a)-8(b) show that MM MSCs have an increased ability to supress activated Macrophages compared to healthy controls.

1-18. (canceled)

19. A method of treating cancer in a subject in need thereof, the method comprising inhibiting or reducing sialylation of specifically Mesenchymal Stromal Cells (MSCs) of the subject.

20. The method as claimed in claim 19 wherein inhibiting or reducing sialylation of specifically MSCs comprises administering a sialyltransferase inhibitor to the subject.

21. The method as claimed in claim 20 wherein the sialyltransferase inhibitor inhibits specifically sialyltransferases associated with or specific to MSCs.

22. The method as claimed in claim 20 wherein the inhibitor is targeted for delivery to specifically bone marrow microenvironment.

23. The method as claimed in claim 20 wherein the inhibitor is targeted for delivery to specifically MSCs.

24. The method as claimed in claim 20 wherein the inhibitor is conjugated to a monoclonal antibody that targets an antigen specific to the bone marrow microenvironment or MSCs.

25. The method as claimed in claim 24 wherein the inhibitor is a sialyltransferase inhibitor selected from the group consisting of (i) sialic acid analogs, (ii) CMP-sialic acid analogs, (iii) cytidine analogs, (iv) oligosaccharide derivatives, (v) aromatic compounds, (vi) flavonoids and (vii) lithocholic acid analogs.

26. The method as claimed in claim 25 wherein the inhibitor is a sialic acid analog.

27. The method as claimed in claim 26 wherein the sialyltransferase inhibitor is 3Fax-Peracetyl Neu5Ac.

28. The method as claimed in claim 19 wherein inhibiting or reducing sialylation of specifically MSCs comprises administering a sialidase to the subject wherein the sialidase is targeted for delivery to specifically bone marrow microenvironment or MSCs.

29. The method as claimed in claim 28 wherein the sialidase is targeted for delivery to specifically bone marrow microenvironment.

30. The method as claimed in claim 28 wherein the sialidase is targeted for delivery to specifically MSCs.

31. The method as claimed in any claim 28 wherein the sialidase is conjugated to a monoclonal antibody that targets an antigen specific to the bone marrow microenvironment or MSCs.

32. The method as claimed in claim 19 wherein inhibiting or reducing sialylation of specifically MSCs comprises administering MSCs to the subject wherein the MSCs have been manipulated to remove sialic acids prior to administration to the subject.

33. The method as claimed in claim 32 wherein manipulation of the MSCs to remove sialic acids comprises removing alpha 2,6 linked sialic acids.

34. A method of treating cancer in a subject in need thereof, the method comprising administering a small molecule or blocking antibody that blocks interaction between Mesenchymal Stromal Cell (MSC)-sialic acids and lectins and/or between MSC-lectins and sialic acid.

35. The method as claimed in claim 34 wherein the small molecule or blocking antibody blocks interactions between MSC-sialic acids and Siglec 7 and/or between MSC Siglec 7 and sialic acid.

36. The method as claimed in claim 34 wherein the small molecule comprises a sialic acid mimetic.
